(19) United States
(12) Patent Application Publication
(10) Pub. No.: US 2007/0258972 A1
(43) Pub. Date: Nov. 8, 2007

(54) Combination of a Histone Deacetylase Inhibitor with a Death Receptor Ligand

(76) Inventors: Peter Wisdom Atadja, Acton, MA (US); Kapil N. Bhalla, Tampa, FL (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/571,734

(22) PCT Filed: Sep. 17, 2004
(86) PCT No.: PCT/EP04/10468

§ 371 Date: Mar. 19, 2007

Related U.S. Application Data
(60) Provisional application No. 60/504,655, filed on Sep. 18, 2003.

(51) Int. Cl. A61K 45/06; A61K 38/00; A61P 35/02; C07C 233/00; A61K 39/395

(52) U.S. Cl. 424/130.1; 514/2; 514/617; 564/161

(57) ABSTRACT

The invention relates to a method of preventing or treating proliferative diseases such as cancer in a mammal, particularly a human, with a combination of pharmaceutical agents which comprises: (a) an HDAI; and (b) a death receptor ligand. The invention further relates to pharmaceutical compositions comprising: (a) an HDAI; (b) death receptor ligand; and (c) a pharmaceutically acceptable carrier. The present invention further relates to a commercial package or product comprising: (a) a pharmaceutical formulation of an HDAI; and (b) a pharmaceutical formulation of death receptor ligand for simultaneous, concurrent, separate or sequential use.

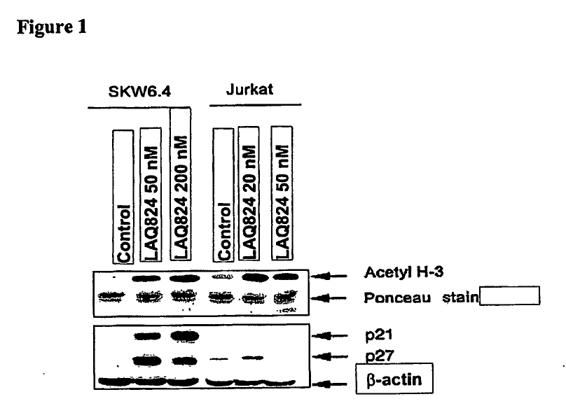

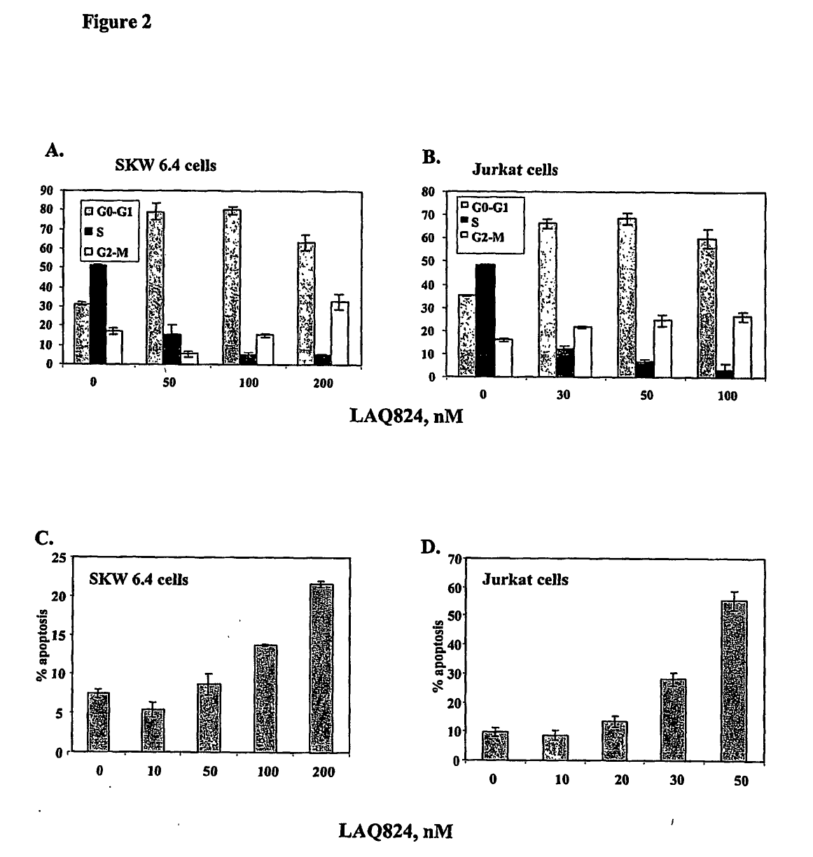

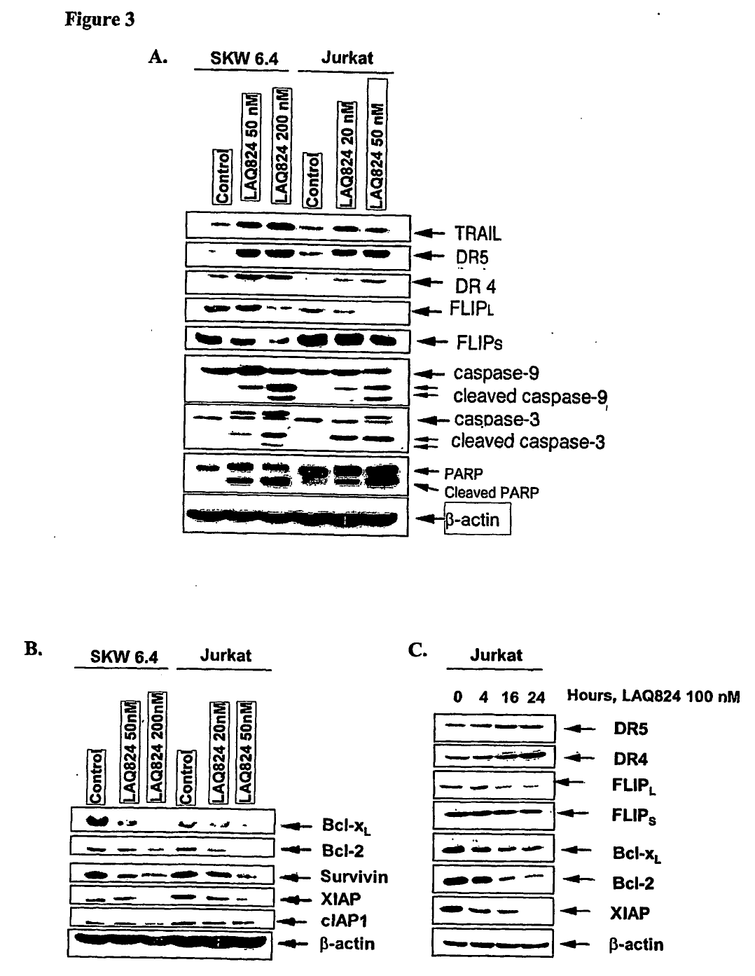

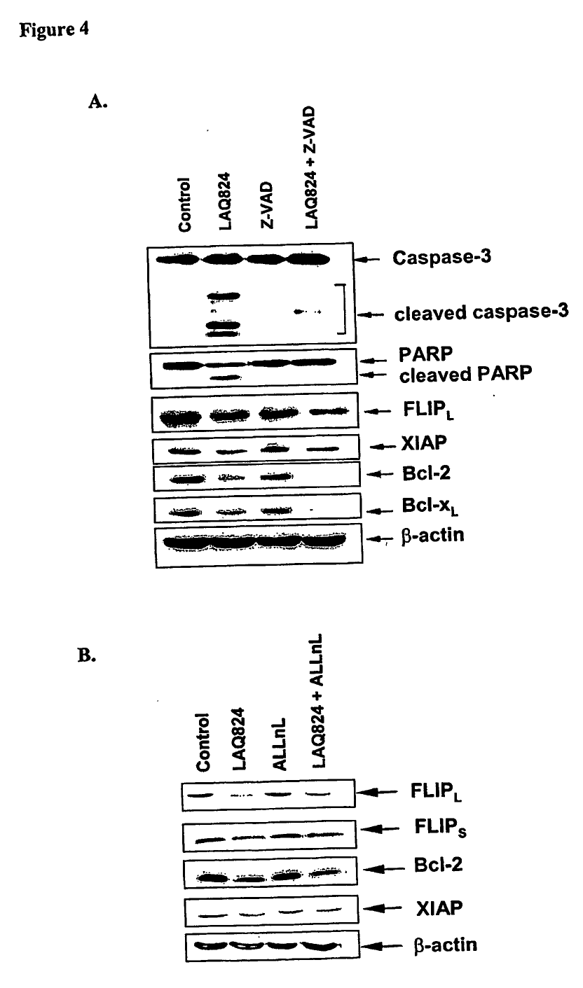

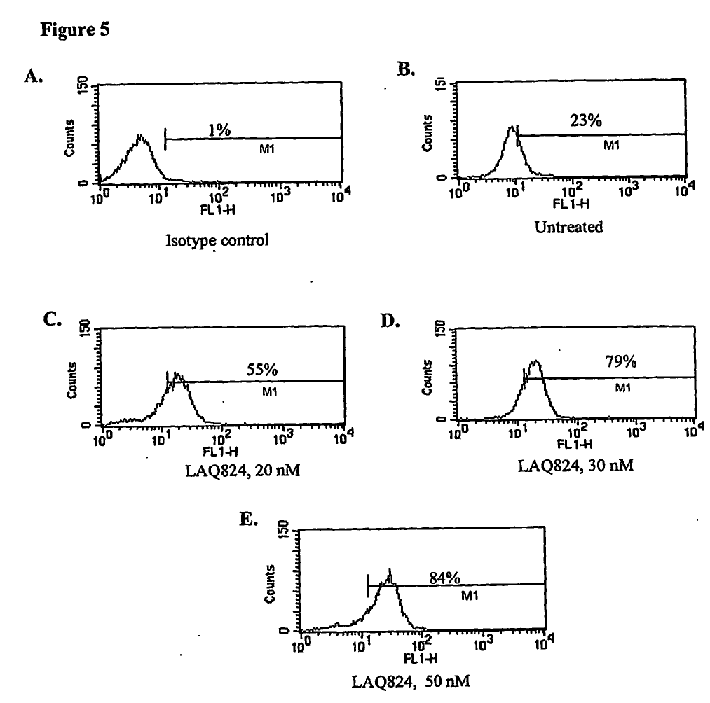

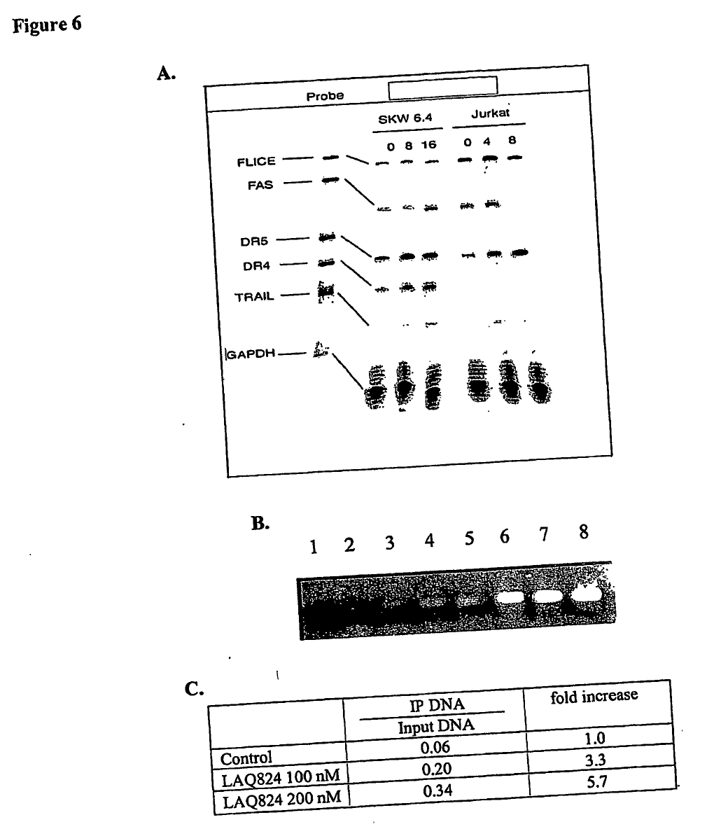

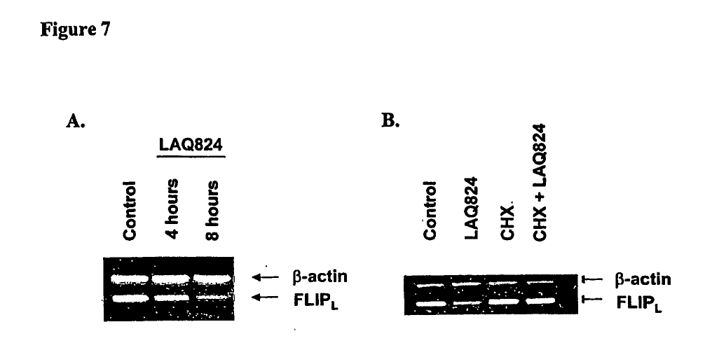

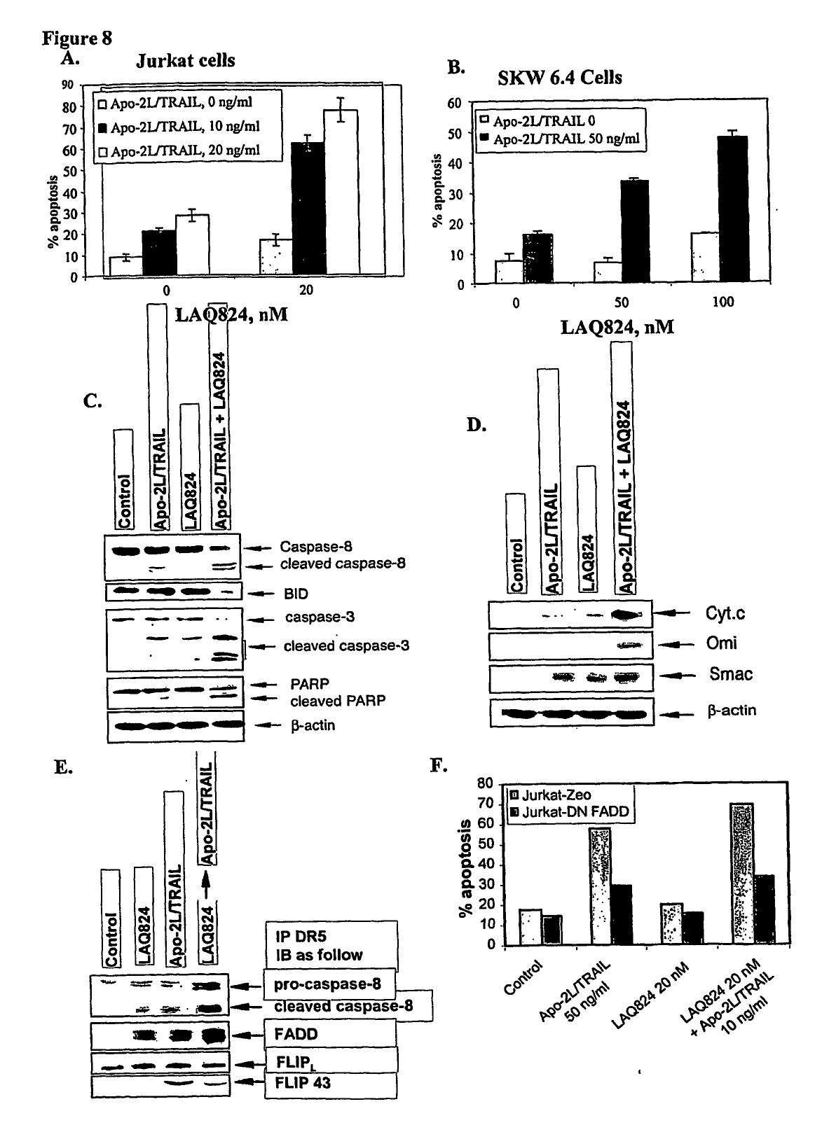

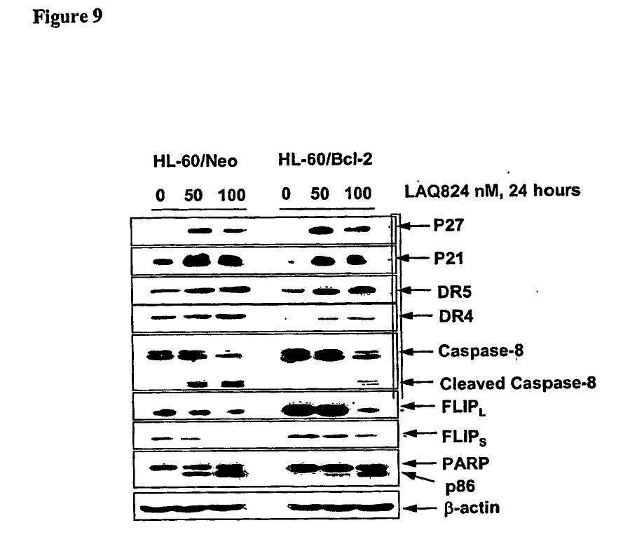

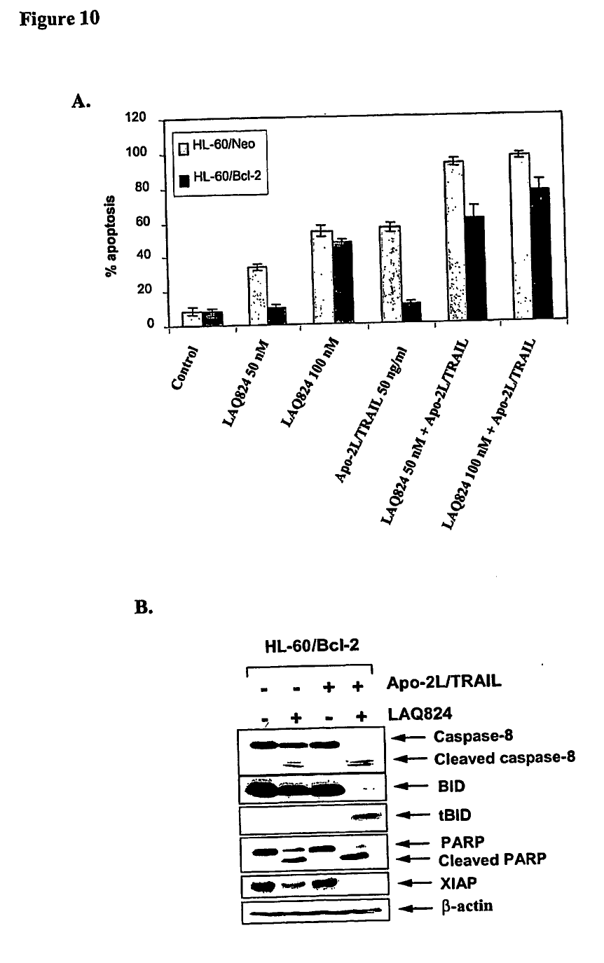

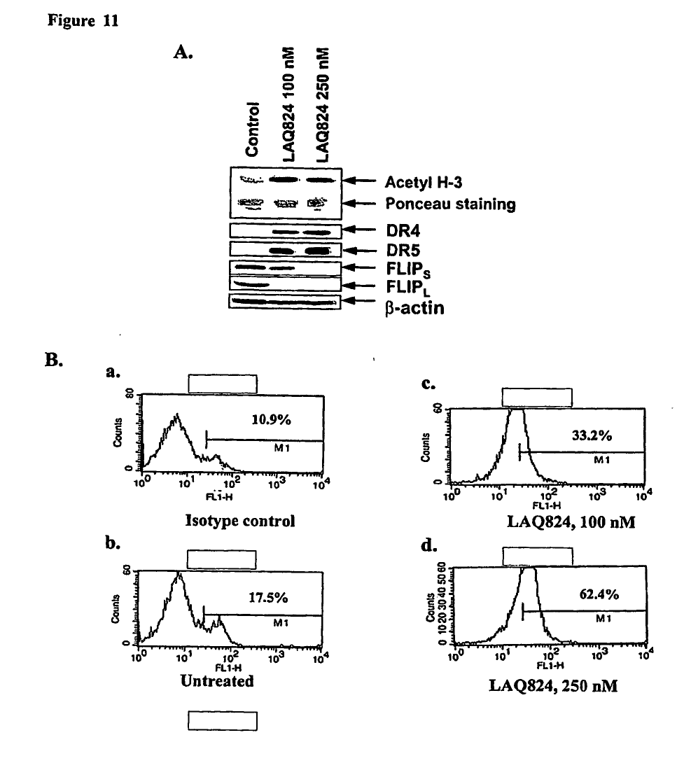

The invention relates to a method of preventing or treating proliferative diseases, such as cancer in a mammal, particularly a human, with a combination of pharmaceutical agents which comprises:
(a) a histone deacetylase inhibitor (HDAI); and
(b) a death receptor ligand.

The invention further relates to pharmaceutical compositions comprising:
(a) an HDAI;
(b) death receptor ligand; and
(c) a pharmaceutically acceptable carrier.

The present invention further relates to a commercial package or product comprising:
(a) a pharmaceutical formulation of an HDAI; and
(b) a pharmaceutical formulation of death receptor ligand for simultaneous, concurrent, separate or sequential use.

### BACKGROUND OF THE INVENTION

Reversible acetylation of histones is a major regulator of gene expression that acts by altering accessibility of transcription factors to DNA. In normal cells, histone deacetylase (HDA) and histone acetyltrasferase together control the level of acetylation of histones to maintain a balance. Inhibition of HDA results in the accumulation of hyperacetylated histones, which results in a variety of cellular responses. Inhibitors of HDA (HDAI) have been studied for their therapeutic effects on cancer cells. Recent developments in the field of HDAI research have provided active compounds, both highly efficacious and stable, that are suitable for treating tumors.

Tumor necrosis factor (TNF)-related apoptosis inducing ligand (TRAIL, also known as Apo-2L) is a member of the TNF family of cytokines that can bind and induce oligomerization of its agonistic cell-membrane death receptors TRAIL-R1 (DR4) and TRAIL-R2 (DR5). Upon binding and cross-linking by Apo-2L/TRAIL, or by agonistic antibodies, the death receptors DR4 and DR5 can trigger the activity of caspase-8 and apoptosis through the assembly of a cell-membrane associated multi-protein death inducing signaling complex (DISC).

### SUMMARY OF THE INVENTION

It has now been shown that treatment with an HDAI induces DR4 and DR5 but represses cFLIP levels, which is associated with increased Apo-2L/TRAIL-induced DISC activity. Co-treatment with an HDAI enhances Apo-2L/TRAIL-induced death inducing signaling complex activity and apoptosis of human acute leukemia cells. This evidence suggests that TRAIL is even more efficacious when used in combination with an HDAI. There are both synergistic and additive advantages, both for efficacy and safety. Therapeutic effects of combinations of an HDAI with TRAIL can result in lower safe dosages ranges of each component in the combination.

The invention relates to a combination for preventing or treating proliferative diseases, such as cancer in a mammal, particularly a human, comprising:
(a) a HDAI; and
(b) a death receptor ligand.

The invention relates to a method of preventing or treating proliferative diseases, such as cancer in a mammal, particularly a human, with a combination of pharmaceutical agents which comprises:
(a) a HDAI; and
(b) a death receptor ligand.

The invention further relates to pharmaceutical compositions comprising:
(a) an HDAI;
(b) death receptor ligand; and
(c) a pharmaceutically acceptable carrier.

The present invention further relates to a commercial package or product comprising:
(a) a pharmaceutical formulation of an HDAI; and
(b) a pharmaceutical formulation of death receptor ligand for simultaneous, concurrent, separate or sequential use.

### DETAILED DESCRIPTION OF THE INVENTION

The Diseases to be Treated

The combinations of the present invention are useful for treating proliferative diseases. A proliferative disease is mainly a tumor disease (or cancer) (and/or any metastases). The inventive compositions are particularly useful for treating a tumor which is a breast cancer, genitourinary cancer, lung cancer, gastrointestinal cancer, epidermoid cancer, melanoma, glioma, ovarian cancer, pancreas cancer, neuroblastoma, head and/or neck cancer or bladder cancer, or in a broader sense renal, brain or gastric cancer; in particular:
(i) a breast tumor; an epidermoid tumor, such as an epidermoid head and/or neck tumor or a mouth tumor; a lung tumor, e.g., a small cell or non-small cell lung tumor; a gastrointestinal tumor, e.g., a colorectal tumor; or a genitourinary tumor, e.g., a prostate tumor, especially a hormone-refractory prostate tumor;
(ii) a proliferative disease that is refractory to the treatment with other chemotherapeutics; or
(iii) a tumor that is refractory to treatment with other chemotherapeutics due to multidrug resistance.

In a broader sense of the invention, a proliferative disease may furthermore be a hyperproliferative condition, such as leukemias (especially acute myeloid leukemia or AML), hyperplasias, fibrosis (especially pulmonary, but also other types of fibrosis, such as renal fibrosis), angiogenesis, psoriasis, atherosclerosis and smooth muscle proliferation in the blood vessels, such as stenosis or restenosis following angioplasty.

Other malignancies which may be treated according to this invention includes a malignancy that is susceptible to treatment with a TRAIL compound.

Where a tumor, a tumor disease, a carcinoma or a cancer are mentioned, also metastasis in the original organ or tissue and/or in any other location are implied alternatively or in addition, whatever the location of the tumor and/or metastasis.

The combinations are selectively toxic or more toxic to rapidly proliferating cells than to normal cells, particularly in human cancer cells, e.g., cancerous tumors, the compound has significant anti proliferative effects and promotes differentiation, e.g., cell cycle arrest and apoptosis. The combinations can induce apoptotic cell death and necrosis.

Death Receptor Ligand

The term “death receptor ligand” as used herein refers to TRAIL, TRAIL/Apo-2L, TRAIL mimetics, agonistic antibodies and other agents that can bind to DR4 and DR5 triggering the activity of caspase-8 and apoptosis through the assembly of a cell-membrane associated multi-protein DISC.

TRAIL has demonstrated the ability to induce apoptosis of certain transformed cells, including a number of different types of cancer cells, as well as virally infected cells. TRAIL is disclosed in U.S. Pat. No. 5,763,223 which is incorporated herein in its entirety. See Wiley et al., “Identification and Characterization of a New Member of the TNF Family that Induces Apoptosis”, Immunity, Vol. 3, pp. 673-682 (1995); and Pitti et al., “Induction of Apoptosis by Apo-2 Ligand, a New Member of the Tumor Necrosis Factor Cytokine Family”, J Biol Chem, Vol. 271, No. 22, pp. 12687-12690 (1996).

The combinations described herein include TRAIL, TRAIL/Apo-2L, TRAIL mimetics, agnostic antibodies and other agents that can bind to DR4 and DR5 triggering the activity of caspase-8 and apoptosis through a cell-membrane associated multi-protein DISC.

The HDAI Compounds

HDAI compounds of particular interest for use in the inventive combination are hydroxamate compounds described by the formula (I): 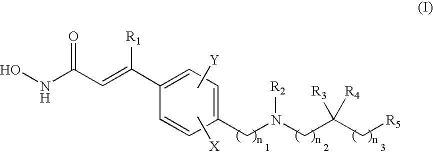
wherein
R₁ is H; halo; or a straight-chain C₁-C₆alkyl, especially methyl, ethyl or n-propyl, which methyl, ethyl and n-propyl substituents are unsubstituted or substituted by one or more substituents described below for alkyl substituents;
R₂ is selected from H; C₁-C₁₀alkyl, preferably C₁-C₆alkyl, e.g., methyl, ethyl or —CH₂CH₂—OH; C₄-C₉cycloalkyl; C₄-C₉heterocycloalkyl; C₄-C₉heterocycloalkylalkyl; cycloalkylalkyl, e.g., cyclopropylmethyl; aryl; heteroaryl; arylalkyl, e.g., benzyl; heteroarylalkyl, e.g., pyridylmethyl; —(CH₂)ₙC(O)R₆; —(CH₂)ₙOC(O)R₆; amino acyl; HON—C(O)—CH═C(R₁)-aryl-alkyl-; and —(CH₂)ₙR₇;
R₃ and R₄ are the same or different and, independently, H; C₁-C₆alkyl; acyl; or acylamino; or
R₃ and R₄, together with the carbon to which they are bound, represent C═O, C═S or C═NR₈; or
R₂, together with the nitrogen to which it is bound, and R₃, together with the carbon to which it is bound, can form a C₄-C₉heterocycloalkyl; a heteroaryl; a polyheteroaryl; a non-aromatic polyheterocycle; or a mixed aryl and non-aryl polyheterocycle ring;
R₅ is selected from H; C₁-C₆alkyl; C₄-C₉cycloalkyl; C₄-C₉heterocycloalkyl; acyl; aryl; heteroaryl; arylalkyl, e.g., benzyl; heteroarylalkyl, e.g., pyridylmethyl; aromatic polycycles; non-aromatic polycycles; mixed aryl and non-aryl polycycles; polyheteroaryl; non-aromatic polyheterocycles; and mixed aryl and non-aryl polyheterocycles;
n, n₁, n₂ and n₃ are the same or different and independently selected from 0-6, when n₁ is 1-6, each carbon atom can be optionally and independently substituted with R₃ and/or R₄;
X and Y are the same or different and independently selected from H; halo; C₁-C₄alkyl, such as CH₃ and CF₃; NO₂; C(O)R₁; OR₉; SR₉; CN; and NR₁₀R₁₁;
R₆ is selected from H; C₁-C₆alkyl; C₄-C₉cycloalkyl; C₄-C₉heterocycloalkyl; cycloalkylalkyl, e.g., cyclopropylmethyl; aryl; heteroaryl; arylalkyl, e.g., benzyl and 2-phenylethenyl; heteroarylalkyl, e.g., pyridylmethyl; OR₁₂; and NR₁₃R₁₄;
R₇ is selected from OR₁₅; SR₁₅; S(O)R₁₆; SO₂R₁₇; NR₁₃R₁₄; and NR₁₂SO₂R₆;
R₈ is selected from H; OR₁₅; NR₁₃R₁₄; C₁-C₆alkyl; C₄-C₉cycloalkyl; C₄-C₉heterocycloalkyl; aryl; heteroaryl; arylalkyl, e.g., benzyl; and heteroarylalkyl, e.g., pyridylmethyl;
R₉ is selected from C₁-C₄alkyl, e.g., CH₃ and CF₃; C(O)-alkyl, e.g., C(O)CH₃; and C(O)CF₃;
R₁₀ and R₁₁ are the same or different and independently selected from H; C₁-C₄alkyl; and —C(O)-alkyl;
R₁₂ is selected from H; C₁-C₆alkyl; C₄-C₉cycloalkyl; C₄-C₉heterocycloalkyl; C₄-C₉heterocycloalkylalkyl; aryl; mixed aryl and non-aryl polycycle; heteroaryl; arylalkyl, e.g., benzyl; and heteroarylalkyl, e.g., pyridylmethyl;
R₁₃ and R₁₄ are the same or different and independently selected from H; C₁-C₆alkyl; C₄-C₉cycloalkyl; C₄-C₉heterocycloalkyl; aryl; heteroaryl; arylalkyl, e.g., benzyl; heteroarylalkyl, e.g., pyridylmethyl; amino acyl; or
R₁₃ and R₁₄, together with the nitrogen to which they are bound, are C₄-C₉heterocycloalkyl; heteroaryl; polyheteroaryl; non-aromatic polyheterocycle; or mixed aryl and non-aryl polyheterocycle;
R₁₅ is selected from H; C₁-C₆alkyl; C₄-C₉cycloalkyl; C₄-C₉heterocycloalkyl; aryl; heteroaryl; arylalkyl; heteroarylalkyl; and (CH₂)ₘZR₁₂;
R₁₆ is selected from C₁-C₆alkyl; C₄-C₉cycloalkyl; C₄-C₉heterocycloalkyl; aryl; heteroaryl; polyheteroaryl; arylalkyl; heteroarylalkyl; and (CH₂)ₘZR₁₂;
R₁₇ is selected from C₁-C₆alkyl; C₄-C₉cycloalkyl; C₄-C₉heterocycloalkyl; aryl; aromatic polycycles; heteroaryl; arylalkyl; heteroarylalkyl; polyheteroaryl and NR₁₃R₁₄;
m is an integer selected from 0-6; and
Z is selected from O; NR₁₃; S; and S(O),
   or a pharmaceutically acceptable salt thereof.

As appropriate, “unsubstituted” means that there is no substituent or that the only substituents are hydrogen.

Halo substituents are selected from fluoro, chloro, bromo and iodo, preferably fluoro or chloro.

Alkyl substituents include straight- and branched-C₁-C₆alkyl, unless otherwise noted. Examples of suitable straight- and branched-C₁-C₆alkyl substituents include methyl, ethyl, n-propyl, 2-propyl, n-butyl, sec-butyl, t-butyl and the like. Unless otherwise noted, the alkyl substituents include both unsubstituted alkyl groups and alkyl groups that are substituted by one or more suitable substituents, including unsaturation, i.e., there are one or more double or triple C—C bonds; acyl; cycloalkyl; halo; oxyalkyl; alkylamino; aminoalkyl; acylamino; and OR₁₅, e.g., alkoxy. Preferred substituents for alkyl groups include halo, hydroxy, alkoxy, oxyalkyl, alkylamino and aminoalkyl.

Cycloalkyl substituents include C₃-C₉cycloalkyl groups, such as cyclopropyl, cyclobutyl, cyclopentyl, cyclohexyl and the like, unless otherwise specified. Unless otherwise noted, cycloalkyl substituents include both unsubstituted cycloalkyl groups and cycloalkyl groups that are substituted by one or more suitable substituents, including C₁-C₆alkyl, halo, hydroxy, aminoalkyl, oxyalkyl, alkylamino and OR₁₅, such as alkoxy. Preferred substituents for cycloalkyl groups include halo, hydroxy, alkoxy, oxyalkyl, alkylamino and aminoalkyl.

The above discussion of alkyl and cycloalkyl substituents also applies to the alkyl portions of other substituents, such as, without limitation, alkoxy, alkyl amines, alkyl ketones, arylalkyl, heteroarylalkyl, alkylsulfonyl and alkyl ester substituents and the like.

Heterocycloalkyl substituents include 3- to 9-membered aliphatic rings, such as 4- to 7-membered aliphatic rings, containing from 1-3 heteroatoms selected from nitrogen, sulfur, oxygen. Examples of suitable heterocycloalkyl substituents include pyrrolidyl, tetrahydrofuryl, tetrahydrothiofuranyl, piperidyl, piperazyl, tetrahydropyranyl, morphilino, 1,3-diazapane, 1,4-diazapane, 1,4-oxazepane and 1,4-oxathiapane. Unless otherwise noted, the rings are unsubstituted or substituted on the carbon atoms by one or more suitable substituents, including C₁-C₆alkyl; C₄-C₉cycloalkyl; aryl; heteroaryl; arylalkyl, e.g., benzyl; heteroarylalkyl, e.g., pyridylmethyl; halo; amino; alkyl amino and OR₁₅, e.g., alkoxy. Unless otherwise noted, nitrogen heteroatoms are unsubstituted or substituted by H, C₁-C₄alkyl; arylalkyl, e.g., benzyl; heteroarylalkyl, e.g., pyridylmethyl; acyl; aminoacyl; alkylsulfonyl; and arylsulfonyl.

Cycloalkylalkyl substituents include compounds of the formula —(CH₂)ₙ₅-cycloalkyl, wherein n5 is a number from 1-6. Suitable alkylcycloalkyl substituents include cyclopentylmethyl, cyclopentylethyl, cyclohexylmethyl and the like. Such substituents are unsubstituted or substituted in the alkyl portion or in the cycloalkyl portion by a suitable substituent, including those listed above for alkyl and cycloalkyl.

Aryl substituents include unsubstituted phenyl and phenyl substituted by one or more suitable substituents including C₁-C₆alkyl; cycloalkylalkyl, e.g., cyclopropylmethyl; O(CO)alkyl; oxyalkyl; halo; nitro; amino; alkylamino; aminoalkyl; alkyl ketones; nitrile; carboxyalkyl; alkylsulfonyl; aminosulfonyl; arylsulfonyl and OR₁₅, such as alkoxy. Preferred substituents include including C₁-C₆alkyl; cycloalkyl, e.g., cyclopropylmethyl; alkoxy; oxyalkyl; halo; nitro; amino; alkylamino; aminoalkyl; alkyl ketones; nitrile; carboxyalkyl; alkylsulfonyl; arylsulfonyl and aminosulfonyl. Examples of suitable aryl groups include C₁-C₄alkylphenyl, C₁-C₄alkoxyphenyl, trifluoromethylphenyl, methoxyphenyl, hydroxyethylphenyl, dimethylaminophenyl, aminopropylphenyl, carbethoxyphenyl, methanesulfonylphenyl and tolylsulfonylphenyl.

Aromatic polycycles include naphthyl, and naphthyl substituted by one or more suitable substituents including C₁-C₆alkyl; alkylcycloalkyl, e.g., cyclopropylmethyl; oxyalkyl; halo; nitro; amino; alkylamino; aminoalkyl; alkyl ketones; nitrile; carboxyalkyl; alkylsulfonyl; arylsulfonyl; aminosulfonyl and OR₁₅, such as alkoxy.

Heteroaryl substituents include compounds with a 5- to 7-membered aromatic ring containing one or more heteroatoms, e.g., from 1-4 heteroatoms, selected from N, O and S. Typical heteroaryl substituents include furyl, thienyl, pyrrole, pyrazole, triazole, thiazole, oxazole, pyridine, pyrimidine, isoxazolyl, pyrazine and the like. Unless otherwise noted, heteroaryl substituents are unsubstituted or substituted on a carbon atom by one or more suitable substituents, including alkyl, the alkyl substituents identified above, and another heteroaryl substituent. Nitrogen atoms are unsubstituted or substituted, e.g., by R₁₃; especially useful N substituents include H, C₁-C₄alkyl, acyl, aminoacyl and sulfonyl.

Arylalkyl substituents include groups of the formula —(CH₂)ₙ₅-aryl, —(CH₂)ₙ₅₋₁—(CH-aryl)-(CH₂)ₙ₅-aryl or —(CH₂)ₙ₅₋₁CH(aryl)(aryl), wherein aryl and n5 are defined above. Such arylalkyl substituents include benzyl, 2-phenylethyl, 1-phenylethyl, tolyl-3-propyl, 2-phenylpropyl, diphenylmethyl, 2-diphenylethyl, 5,5-dimethyl-3-phenylpentyl and the like. Arylalkyl substituents are unsubstituted or substituted in the alkyl moiety or the aryl moiety or both as described above for alkyl and aryl substituents.

Heteroarylalkyl substituents include groups of the formula —(CH₂)ₙ₅-heteroaryl, wherein heteroaryl and n5 are defined above and the bridging group is linked to a carbon or a nitrogen of the heteroaryl portion, such as 2-, 3- or 4-pyridylmethyl, imidazolylmethyl, quinolylethyl and pyrrolylbutyl. Heteroaryl substituents are unsubstituted or substituted as discussed above for heteroaryl and alkyl substituents.

Amino acyl substituents include groups of the formula —C(O)—(CH₂)ₙ—C(H)(NR₁₃R₁₄)—(CH₂)ₙ—R₅, wherein n, R₁₃, R₁₄ and R₅ are described above. Suitable aminoacyl substituents include natural and non-natural amino acids; such as glycinyl, D-tryptophanyl, L-lysinyl, D- or L-homoserinyl, 4-aminobutryic acyl and ±3-amin-4-hexenoyl.

Non-aromatic polycycle substituents include bicyclic and tricyclic fused ring systems where each ring can be 4- to 9-membered and each ring can contain zerio, one or more double and/or triple bonds. Suitable examples of non-aromatic polycycles include decalin, octahydroindene, perhydrobenzocycloheptene and perhydrobenzo-[f]-azulene. Such substituents are unsubstituted or substituted as described above for cycloalkyl groups.

Mixed aryl and non-aryl polycycle substituents include bicyclic and tricyclic fused ring systems where each ring can be 4- to 9-membered and at least ore ring is aromatic. Suitable examples of mixed aryl and non-aryl polycycles include methylenedioxyphenyl, bis-methylenedioxyphenyl, 1,2,3,4-tetrahydronaphthalene, dibenzosuberane, dihdydroanthracene and 9H-fluorene. Such substituents are unsubstituted or substituted by nitro or as described above for cycloalkyl groups.

Polyheteroaryl substituents include bicyclic and tricyclic fused ring systems where each ring can independently be 5 or 6 membered and contain one or more heteroatom, for example, 1, 2, 3, or 4 heteroatoms, chosen from O, N or S such that the fused ring system is aromatic. Suitable examples of polyheteroaryl ring systems include quinoline, isoquinoline, pyridopyrazine, pyrrolopyridine, furopyridine, indole, benzofuran, benzothiofuran, benzindole, benzoxazole, pyrroloquinoline, and the like. Unless otherwise noted, polyheteroaryl substituents are unsubstituted or substituted on a carbon atom by one or more suitable substituents, including alkyl, the alkyl substituents identified above and a substituent of the formula —O—(CH₂CH═CH(CH₃)(CH₂))₁₋₃H. Nitrogen atoms are unsubstituted or substituted, e.g., by R₁₃, especially useful N substituents include H, C₁-C₄alkyl, acyl, aminoacyl and sulfonyl.

Non-aromatic polyheterocyclic substituents include bicyclic and tricyclic fused ring systems where each ring can be 4- to 9-membered, contain one or more heteroatom, e.g., 1, 2, 3 or 4 heteroatoms, chosen from O, N or S and contain zero or one or more C—C double or triple bonds. Suitable examples of non-aromatic polyheterocycles include hexitol, cis-perhydro-cyclohepta[b]pyridinyl, decahydro-benzo[f][1,4]oxazepinyl, 2,8-dioxabicyclo[3.3.0]octane, hexahydro-thieno[3,2-b]thiophene, perhydropyrrolo[3,2-b]pyrrole, perhydronaphthyridine, perhydro-1H-dicyclopenta[b,e]pyran. Unless otherwise noted, non-aromatic polyheterocyclic substituents are unsubstituted or substituted on a carbon atom by one or more substituents, including alkyl and the alkyl substituents identified above. Nitrogen atoms are unsubstituted or substituted, e.g., by R₁₃, especially useful N substituents include H, C₁-C₄alkyl, acyl, aminoacyl and sulfonyl.

Mixed aryl and non-aryl polyheterocycles substituents include bicyclic and tricyclic fused ring systems where each ring can be 4- to 9-membered, contain one or more heteroatom chosen from O, N or S, and at least one of the rings must be aromatic. Suitable examples of mixed aryl and non-aryl polyheterocycles include 2,3-dihydroindole, 1,2,3,4-tetrahydroquinoline, 5,11-dihydro-10H-dibenz[b,e][1,4]diazepine, 5H-dibenzo[b,e][1,4]diazepine, 1,2-dihydropyrrolo[3,4-b][1,5]benzodiazepine, 1, 5-dihydro-pyrido[2,3-b][1,4]diazepin-4-one, 1,2,3,4,6,11-hexahydro-benzo[b]pyrido[2,3-e][1,4]diazepin-5-one. Unless otherwise noted, mixed aryl and non-aryl polyheterocyclic substituents are unsubstituted or substituted on a carbon atom by one or more suitable substituents including —N—OH, ═N—OH, alkyl and the alkyl substituents identified above. Nitrogen atoms are unsubstituted or substituted, e.g., by R₁₃; especially useful N substituents include H, C₁-C₄alkyl, acyl, aminoacyl and sulfonyl.

Amino substituents include primary, secondary and tertiary amines and in salt form, quaternary amines. Examples of amino substituents include mono- and di-alkylamino, mono- and di-aryl amino, mono- and di-arylalkyl amino, aryl-arylalkylamino, alkyl-arylamino, alkyl-arylalkylamino and the like.

Sulfonyl substituents include alkylsulfonyl and arylsulfonyl, e.g., methane sulfonyl, benzene sulfonyl, tosyl and the like.

Acyl substituents include groups of formula —C(O)—W, —OC(O)—W, —C(O)—O—W or —C(O)NR₁₃R₁₄, where W is R₁₆, H or cycloalkylalkyl.

Acylamino substituents include substituents of the formula —N(R₁₂)C(O)—W, —N(R₁₂)C(O)—O—W and —N(R₁₂)C(O)—NHOH and R₁₂ and W are defined above.

The R₂ substituent HON—C(O)—CH═C(R₁)-aryl-alkyl- is a group of the formula 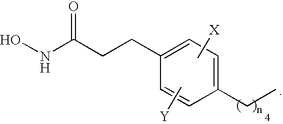
Preferences for each of the substituents include the following:
R₁ is H, halo or a straight-chain C₁-C₄ alkyl;
R₂ is selected from H, C₁-C₆alkyl, C₄-C₉cycloalkyl, C₄-C₉heterocycloalkyl, cycloalkylalkyl, aryl, heteroaryl, arylalkyl, heteroarylalkyl, —(CH₂)ₙC(O)R₆, amino acyl and —(CH₂)ₙR₇;
R₃ and R₄ are the same or different and independently selected from H and C₁-C₆alkyl; or
R₃ and R₄ together with the carbon to which they are bound represent C═O, C═S or C═NR₈;
R₅ is selected from H, C₁-C₆alkyl, C₄-C₉cycloalkyl, C₄-C₉heterocycloalkyl, aryl, heteroaryl, arylalkyl, heteroarylalkyl, a aromatic polycycle, a non-aromatic polycycle, a mixed aryl and non-aryl polycycle, polyheteroaryl, a non-aromatic polyheterocycle, and a mixed aryl and non-aryl polyheterocycle;
n, n₁, n₂ and n₃ are the same or different and independently selected from 0-6, when n₁ is 1-6, each carbon atom is unsubstituted or independently substituted with R₃ and/or R₄;
X and Y are the same or different and independently selected from H, halo, C₁-C₄alkyl, CF₃, NO₂, C(O)R₁, OR₉, SR₉, CN and NR₁₀R₁₁;
R₆ is selected from H, C₁-C₆alkyl, C₄-C₉cycloalkyl, C₄-C₉heterocycloalkyl, alkylcycloalkyl, aryl, heteroaryl, arylalkyl, heteroarylalkyl, OR₁₂ and NR₁₃R₁₄;
R₇ is selected from OR₁₅, SR₁₅, S(O)R₁₆, SO₂R₁₇, NR₁₃R₁₄ and NR₁₂SO₂R₆;
R₈ is selected from H, OR₁₅, NR₁₃R₁₄, C₁-C₆alkyl, C₄-C₉cycloalkyl, C₄-C₉heterocycloalk aryl, heteroaryl, arylalkyl and heteroarylalkyl;
R₉ is selected from C₁-C₄alkyl and C(O)-alkyl;
R₁₀ and R₁₁ are the same or different and independently selected from H, C₁-C₄alkyl and —C(O)-alkyl;
R₁₂ is selected from H, C₁-C₆alkyl, C₄-C₉cycloalkyl, C₄-C₉heterocycloalkyl, aryl, heteroaryl, arylalkyl and heteroarylalkyl;
R₁₃ and R₁₄ are the same or different and independently selected from H, C₁-C₆alkyl, C₄-C₉cycloalkyl, C₄-C₉heterocycloalkyl, aryl, heteroaryl, arylalkyl, heteroarylalkyl and amino acyl;
R₁₅ is selected from H, C₁-C₆alkyl, C₄-C₉cycloalkyl, C₄-C₉heterocycloalkyl, aryl, heteroaryl, arylalkyl, heteroarylalkyl and (CH₂)ₘZR₁₂;
R₁₆ is selected from C₁-C₆alkyl, C₄-C₉cycloalkyl, C₄-C₉heterocycloalkyl, aryl, heteroaryl, arylalkyl, heteroarylalkyl and (CH₂)ₘZR₁₂;
R₁₇ is selected from C₁-C₆alkyl, C₄-C₉cycloalkyl, C₄-C₉heterocycloalkyl, aryl, heteroaryl, arylalkyl, heteroarylalkyl and NR₁₃R₁₄;
m is an integer selected from 0-6; and
Z is selected from O, NR₁₃, S and S(O);
   or a pharmaceutically acceptable salt thereof.

Useful compounds of the formula (I), include those wherein each of R₁, X, Y, R₃ and R₄ is H, including those wherein one of n₂ and n₃ is 0 and the other is 1, especially those wherein R₂ is H or —CH₂—CH₂—OH.

One suitable genus of hydroxamate compounds are those of formula (Ia) 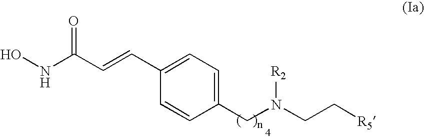
wherein
n₄ is 0-3;
R₂ is selected from H, C₁-C₆alkyl, C₄-C₉cycloalkyl, C₄-C₉heterocycloalkyl, cycloalkylalkyl, aryl, heteroaryl, arylalkyl, heteroarylalkyl, —(CH₂)ₙC(O)R₆, amino acyl and —(CH₂)ₙR₇; and
R′₅ is heteroaryl; heteroarylalkyl, e.g., pyridylmethyl; aromatic polycycles; non-aromatic polycycles; mixed aryl and non-aryl polycycles; polyheteroaryl or mixed aryl; and non-aryl polyheterocycles;
   or a pharmaceutically acceptable salt thereof.

Another suitable genus of hydroxamate compounds are those of formula (Ia) 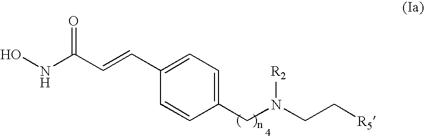
wherein
n₄ is 0-3;
R₂ is selected from H, C₁-C₆alkyl, C₄-C₉cycloalkyl, C₄-C₉heterocycloalkyl, cycloalkylalkyl, aryl, heteroaryl, arylalkyl, heteroarylalkyl, —(CH₂)ₙC(O)R₆, amino acyl and —(CH₂)ₙR₇;
R′₅ is aryl; arylalkyl; aromatic polycycles; non-aromatic polycycles and mixed aryl; and non-aryl polycycles, especially aryl, such as p-fluorophenyl, p-chlorophenyl, p-O—C₁-C₄alkylphenyl, such as p-methoxyphenyl, and p-C₁-C₄alkylphenyl; and arylalkyl, such as benzyl, ortho-, meta- or para-fluorobenzyl, ortho-, meta- or para-chlorobenzyl, ortho-, meta- or para-mono, di- or tri-O—C₁-C₄alkylbenzyl, such as ortho-, meta- or para-methoxybenzyl, m,p-diethoxybenzyl, o,m,p-triimethoxybenzyl and ortho-, meta- or para-mono, di- or tri-C₁-C₄alkylphenyl, such as p-methyl, m,m-diethylphenyl;
   or a pharmaceutically acceptable salt thereof.

Another interesting genus is the compounds of formula (Ib) 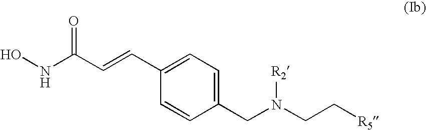
wherein
R′₂ is selected from H; C₁-C₆alkyl; C₄-C₆cycloalkyl; cycloalkylalkyl, e.g., cyclopropylmethyl; (CH₂)₂₋₄OR₂₁, where R₂₁ is H, methyl, ethyl, propyl and i-propyl; and
R″₅ is unsubstituted 1H-indol-3-yl, benzofuran-3-yl or quinolin-3-yl, or substituted 1H-indol-3-yl, such as 5-fluoro-1H-indol-3-yl or 5-methoxy-1H-indol-3-yl, benzofuran-3-yl or quinolin-3-yl;
   or a pharmaceutically acceptable salt thereof.

Another interesting genus of hydroxamate compounds are the compounds of formula (Ic) 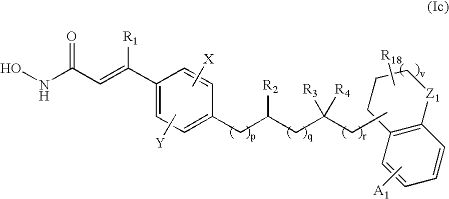
wherein
the ring containing Z₁ is aromatic or non-aromatic, which non-aromatic rings are saturated or unsaturated,
Z₁ is O, S or N—R₂₀;
R₁₈ is H; halo; C₁-C₆alkyl (methyl, ethyl, t-butyl); C₃-C₇cycloalkyl; aryl, e.g., unsubstituted phenyl or phenyl substituted by 4-OCH₃ or 4-CF₃; or heteroaryl, such as 2-furanyl, 2-thiophenyl or 2-, 3- or 4-pyridyl;
R₂₀ is H; C₁-C₆alkyl; C₁-C₆alkyl-C₃-C₉cycloalkyl, e.g., cyclopropylmethyl; aryl; heteroaryl; arylalkyl, e.g., benzyl; heteroarylalkyl, e.g., pyridylmethyl; acyl, e.g., acetyl, propionyl and benzoyl; or sulfonyl, e.g., methanesulfonyl, ethanesulfonyl, benzenesulfonyl and toluenesulfonyl;
A₁ is 1, 2 or 3 substituents which are independently H; C₁-C₆alkyl; —OR₁₉; halo; alkylamino; aminoalkyl; halo; or heteroarylalkyl, e.g., pyridylmethyl;
R₁₉ is selected from H; C₁-C₆alkyl; C₄-C₉cycloalkyl; C₄-C₉heterocycloalkyl; aryl; heteroaryl; arylalkyl, e.g., benzyl; heteroarylalkyl, e.g., pyridylmethyl and —(CH₂CH═CH(CH₃)(CH₂))₁₋₃H;
R₂ is selected from H, C₁-C₆alkyl, C₄-C₉cycloalkyl, C₄-C₉heterocycloalkyl, cycloalkylalkyl, aryl, heteroaryl, arylalkyl, heteroarylalkyl, —(CH₂)ₙC(O)R₆, amino acyl and —(CH₂)ₙR₇;
v is 0, 1 or 2;
p is 0-3; and
q is 1-5 and r is 0; or
q is 0 and r is 1-5;
   or a pharmaceutically acceptable salt thereof. The other variable substituents are as defined above.

Especially useful compounds of formula (Ic), are those wherein R₂ is H, or —(CH₂)ₚCH₂OH, wherein p is 1-3, especially those wherein R₁ is H; such as those wherein R₁ is H and X and Y are each H, and wherein q is 1-3 and r is 0 or wherein q is 0 and r is 1-3, especially those wherein Z₁ is N—R₂₀. Among these compounds R₂ is preferably H or —CH₂—CH₂—OH and the sum of q and r is preferably 1.

Another interesting genus of hydroxamate compounds are the compounds of formula (Id) 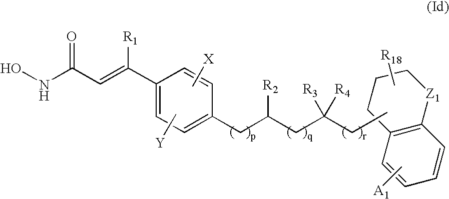
wherein
Z₁ is O, S or N—R₂₀;
R₁₈ is H; halo; C₁-C₆alkyl (methyl, ethyl, t-butyl); C₃-C₇cycloalkyl; aryl, e.g., unsubstituted phenyl or phenyl substituted by 4-OCH₃ or 4-CF₃; or heteroaryl;
R₂₀ is H; C₁-C₆alkyl, C₁-C₆alkyl-C₃-C₉cycloalkyl, e.g., cyclopropylmethyl; aryl; heteroaryl; arylalkyl, e.g., benzyl; heteroarylalkyl, e.g., pyridylmethyl; acyl, e.g., acetyl, propionyl and benzoyl; or sulfonyl, e.g., methanesulfonyl, ethanesulfonyl, benzenesulfonyl, toluenesulfonyl);
A₁ is 1, 2 or 3 substituents which are independently H, C₁-C₆alkyl, —OR₁₉ or halo;
R₁₉ is selected from H; C₁-C₆alkyl; C₄-C₉cycloalkyl; C₄-C₉heterocycloalkyl; aryl; heteroaryl; arylalkyl, e.g., benzyl; and heteroarylalkyl, e.g., pyridylmethyl;
p is 0-3; and
q is 1-5 and r is 0; or
q is 0 and r is 1-5;
   or a pharmaceutically acceptable salt thereof. The other variable substituents are as defined above.

Especially useful compounds of formula (Id), are those wherein R₂ is H or —(CH₂)ₚCH₂OH, wherein p is 1-3, especially those wherein R₁ is H; such as those wherein R₁ is H and X and Y are each H, and wherein q is 1-3 and r is 0 or wherein q is 0 and r is 1-3. Among these compounds R₂ is preferably H or —CH₂—CH₂—OH and the sum of q and r is preferably 1.

The present invention further relates to compounds of the formula (Ie) 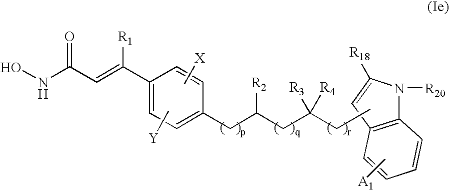
or a pharmaceutically acceptable salt thereof. The variable substituents are as defined above.

Especially useful compounds of formula (Ie), are those wherein R₁₈ is H, fluoro, chloro, bromo, a C₁-C₄alkyl group, a substituted C₁-C₄alkyl group, a C₃-C₇cycloalkyl group, unsubstituted phenyl, phenyl substituted in the para position, or a heteroaryl, e.g., pyridyl, ring.

Another group of useful compounds of formula (Ie), are those wherein R₂ is H or —(CH₂)ₚCH₂OH, wherein p is 1-3, especially those wherein R₁ is H; such as those wherein R₁ is H and X and Y are each H, and wherein q is 1-3 and r is 0 or wherein q is 0 and r is 1-3. Among these compounds R₂ is preferably H or —CH₂—CH₂—OH and the sum of q and r is preferably 1. Among these compounds p is preferably 1 and R₃ and R₄ are preferably H.

Another group of useful compounds of formula (Ie), are those wherein R₁₈ is H, methyl, ethyl, t-butyl, trifluoromethyl, cyclohexyl, phenyl, 4-methoxyphenyl, 4-trifluoromethylphenyl, 2-furanyl, 2-thiophenyl, or 2-, 3- or 4-pyridyl wherein the 2-furanyl, 2-thiophenyl and 2-, 3- or 4-pyridyl substituents are unsubstituted or substituted as described above for heteroaryl rings; R₂ is H or —(CH₂)ₚCH₂OH, wherein p is 1-3; especially those wherein R₁ is H and X and Y are each H, and wherein q is 1-3 and r is 0 or wherein q is 0 and r is 1-3. Among these compounds R₂ is preferably H or —CH₂—CH₂—OH and the sum of q and r is preferably 1.

Those compounds of formula (Ie), wherein R₂₀ is H or C₁-C₆alkyl, especially H, are important members of each of the subgenuses of compounds of formula (Ie) described above.

N-hydroxy-3-[4-[[(2-hydroxyethyl)[2-(1H-indol-3-yl)ethyl]-amino]methyl]phenyl]-2E-2-propenamide, N-hydroxy-3-[4-[[[2-(1H-indol-3-yl)ethyl]-amino]methyl]phenyl]-2E-2-propenamide and N-hydroxy-3-[4-[[[2-(2-methyl-1H-indol-3-yl)-ethyl]-amino]methyl]phenyl]-2E-2-propenamide or a pharmaceutically acceptable salt thereof, are important compounds of formula (Ie).

The present invention further relates to the compounds of the formula (If) 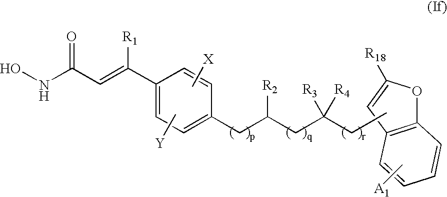
or a pharmaceutically acceptable salt thereof. The variable substituents are as defined above.

Useful compounds of formula (If), are include those wherein R₂ is H or —(CH₂)ₚCH₂OH, wherein p is 1-3, especially those wherein R₁ is H; such as those wherein R₁ is H and X and Y are each H, and wherein q is 1-3 and r is 0 or wherein q is 0 and r is 1-3. Among these compounds R₂ is preferably H or —CH₂—CH₂—OH and the sum of q and r is preferably 1.

N-hydroxy-3-[4-[[[2-(benzofur-3-yl)-ethyl]-amino]methyl]phenyl]-2E-2-propenamide or a pharmaceutically acceptable salt thereof, is an important compound of formula (If).

The compounds described above are often used in the form of a pharmaceutically acceptable salt. Pharmaceutically acceptable salts include, when appropriate, pharmaceutically acceptable base addition salts and acid addition salts, for example, metal salts, such as alkali and alkaline earth metal salts, ammonium salts, organic amine addition salts and amino acid addition salts and sulfonate salts. Acid addition salts include inorganic acid addition salts, such as hydrochloride, sulfate and phosphate; and organic acid addition salts, such as alkyl sulfonate, arylsulfonate, acetate, maleate, fumarate, tartrate, citrate and lactate. Examples of metal salts are alkali metal salts, such as lithium salt, sodium salt and potassium salt; alkaline earth metal salts, such as magnesium salt and calcium salt, aluminum salt and zinc salt. Examples of ammonium salts are ammonium salt and tetramethylammonium salt. Examples of organic amine addition salts are salts with morpholine and piperidine. Examples of amino acid addition salts are salts with glycine, phenylalanine, glutamic acid and lysine. Sulfonate salts include mesylate, tosylate and benzene sulfonic acid salts.

Additional HDAI compounds within the scope of formula (I), and their synthesis, are disclosed in WO 02/22577 published Mar. 21, 2002 which is incorporated herein by reference in its entirety.

The Combinations

Thus, in a first aspect, the present invention relates to a combination comprising pharmaceutically effective amounts of a combination of:
(a) an HDAI of formula (I); and
(b) a death receptor ligand.

In another aspect, the present invention relates to a method for the prevention of treatment of proliferative diseases, such as cancer in a mammal, preferably a human patient, which comprises treating the patient concurrently or sequentially with pharmaceutically effective amounts of a combination of:
(a) an HDAI of formula (I); and
(b) a death receptor ligand.

In a specific embodiment, the inventive method is a method for the prevention or treatment of leukemia. In another embodiment, the inventive method is a method for the prevention and treatment of AML.

According to the present invention, a patient is treated concurrently or sequentially with therapeutically effective amounts of an HDAI and a death receptor ligand in order to prevent or treat proliferative diseases, such as cancer, according to a dosage regimen that is appropriate for the individual agent. For example, the HDAI may be administered once or more daily and the death receptor ligand may be administered once daily, on alternate days or on some other schedule, as is appropriate for the death receptor ligand when used without the HDAI. One of skill in the art has the ability to determine appropriate pharmaceutically effective amounts of the combination components. The HDAI is administered at an appropriate dose in the range from 100-1500 mg daily, e.g., 200-1000 mg/day, such as 200, 400, 500, 600, 800, 900 or 1000 mg/day, administered in one or two doses daily. Appropriate dosages and the frequency of administration of the death receptor ligand will depend on such factors, as the nature and severity of the indication being treated, the desired response, the condition of the patient and so forth.

The compounds or the pharmaceutically acceptable salts thereof, are administered locally, by intravenous injection, continuous infusion, sustained-release from implants, parenteral injections or other suitable technique. They may also be administered as an oral pharmaceutical formulation in the form of a tablet, capsule or syrup.

The present invention further relates to “a combined preparation”, which, as used herein, defines especially a “kit of parts” in the sense that the combination partners (a) and (b), as defined above, can be dosed independently or by use of different fixed combinations with distinguished amounts of the combination partners (a) and (b), i.e., simultaneously or at different time points. The parts of the kit of parts can then, e.g., be administered simultaneously or chronologically staggered, that is at different time points and with equal or different time intervals for any part of the kit of parts. The two agents may be administered via the same route, or different routes may be used. The ratio of the total amounts of the combination partner (a) to the combination partner (b) to be administered in the combined preparation can be varied, e.g., in order to cope with the needs of a patient sub-population to be treated or the needs of the single patient based on the severity of any side effects that the patient experiences.

The combination partner (a) or (b) or a pharmaceutically acceptable salt thereof may also be used in form of a hydrate or other solvate.

It is one objective of this invention to provide a pharmaceutical composition comprising a quantity, which is therapeutically effective against proliferative diseases including pre-malignant lesions, as well as both solid and undifferentiated malignancies, such as pre-malignant colon lesions or colon cancer or other malignancy comprising the combination of the invention. In this composition, the combination partners (a) and (b) can be administered together, one after the other or separately in one combined unit dosage form or in two separate unit dosage forms. The unit dosage form may also be a fixed combination.

The following Example illustrates the invention described above; it is not, however, intended to limit the scope of the invention in any way. The beneficial effects of the combination of the invention can also be determined by other test models known as such to the person skilled in the pertinent art.

### EXAMPLES

Methods

Reagents

LAQ824 is provided by Novartis Pharmaceuticals Inc. (East Hanover, N.J.). The recombinant human trimeric form of Apo-2L/TRAIL is from Genentech, Inc. (South San Francisco, Calif.) and is produced in E. coli. Anti-Bid and anti-Smac/DIABLO antibodies are provided by Dr. Xiaodong Wang of the University of Texas, Southwestern School of Medicine (Dallas, Tex.). Monoclonal anti-XIAP antibody is purchased from Boehringer Mannheim (Indianapolis, Ind.). Polyclonal anti-PARP and monoclonal anti-clAP-1, caspase-9 and caspase-3 antibodies are purchased from Pharmingen Inc. (San Diego, Calif.). Polyclonal anti-caspase-8 antibody is purchased from Upstate Biotechnology (Lake Placid, N.Y.), while monoclonal anti-survivin is purchased from Alpha Diagnostic (San Antonio, Tex.). DR4 antibody is purchased from Alexis Corp. (San Diego, Calif.). Polyclonal anti-DR5 is obtained from Cayman Chemicals Co. (Ann Arbor, Mich.). The antibodies for the immunoblot analyses to detect the levels of p21 and p27 are obtained, as previously described. Monoclonal anti-cytochrome oxidase-2 antibody is purchased from Molecular Probe (Eugene, OR). z-VAD-FMK and LLnL are purchased from Calbiochem (San Diego, Calif.).

Cells

Jurkat T cell leukemia and SKW6.4 B lymphoblast cells are obtained from American Tissue Culture Collection (Manassas, Va.). HL-60/Bcl-2 cells with ectopic over-expression of Bcl-2 and the control HL-60/Neo cells are created and maintained in culture as previously described. Primary leukemia blasts from six patients with AML in relapse are harvested, as previously described, from the peripheral blood or bone marrow after informed consent, as a part of a protocol study sanctioned by the local Institutional Review Board. The purity of leukemia blasts in the samples prior to culture in LAQ824 and/or Apo-2L/TRAIL was at least 80% or more, as determined by morphologic evaluation after Wright staining.

Flow Cytometry Analysis of Cell Cycle Status

The flow cytometric evaluation of the cell cycle status is performed according to a previously described method. The percentage of cells in the G1, S-phase, and G2/M phases are calculated using Multicycle software (Phoenix Flow Systems, San Diego, Calif.).

Apoptosis Assessment by Annexin-V Staining

After drug treatments, cells are resuspended in 100 μL of the staining solution containing annexin-V fluorescein and propidium iodide in a HEPES buffer (Annexin-V-FLUOS Staining Kit, Boehringer-Mannheim, and Indianapolis, Ind.). Following incubation at room temperature for 15 minutes, annexin V positive cells are estimated by flow cytometry, as previously described.

Morphologic Assessment of Apoptosis

After drug treatment, 50×10³ cells are washed and resuspended in PBS (pH 7.3). Cytospun preparations of the cell suspensions are fixed and stained with Wright stain. Cell morphology was determined by light microscopy. In all, five different fields are randomly selected for counting of at least 500 cells. The percentage of apoptotic cells was calculated for each experiment, as described previously.

Preparation of S-100 and Western Analysis of Cytosolic Cytochrome c (cyt c), Smac and Omi

Untreated and drug-treated cells are harvested by centrifugation at 1,000×g for 10 minutes at 4° C. The cell pellets are washed once with ice-cold PBS and re-suspended with 5 volumes of buffer (20 mM HEPES-KOH, pH 7.5, 10 mM KCl, 1.5 mM MgCl2, 1 mM sodium EDTA, 1 mM sodium EGTA, 1 mM dithiothreitol and 0.1 mM PMSF), containing 250 mM sucrose. The cells are homogenized with a 22-gauge needle, and the homogenates are centrifuged at 10,000×g for 10 minutes at 4° C. The supernatants are further centrifuged at 100,000×g for 30 minutes. The resulting supernatants (S-100) are collected and the protein concentrations are determined by using the BCA protein assay reagent from Pierce Biotechnology Inc. (Rockford, Ill.). A total of 75 μg of the S-100 fraction was used for Western blot analysis of cyt c, Smac and Omi/HtrA2.

Western Analyses of Proteins

Western analyses of DR4, DR5, Apo-2L, FADD, Caspase-8, c-FLIPL & S, BID, Caspase-9, Caspase-3, PARP, XIAP, clAP1, survivin and β-actin are performed using specific anti-sera or monoclonal antibodies according to previously reported protocols. Horizontal scanning densitometry was performed on Western blots by using acquisition into Adobe Photo Shop (Apple, Inc., Cupertino, Calif.) and analysis by the NIH Image Program (U.S. National Institutes of Health, Bethesda, Md.). The expression of β-actin was used as a control.

Apo-2L/TRAIL-Induced DISC Analysis

Untreated or LAQ824 treated SKW 6.4 or Jurkat cells are suspended at a final concentration of 10⁶ cells/mL in a pre-warmed, complete RPMI media. Cells are treated with 100 ng/mL Apo-2L/TRAIL for 2 hours at 37° C, followed by washing with 1 mL of ice-cold PBS. Cells are lysed in 500 μL lysis buffer (25 mM Tris-HCl, pH 7.2, 150 mM NaCl, 25 mM NaF, 1 mM benzamidine, 1.0% Triton X-100, 2 μg/mL aprotinin 2 μg/mL leupeptin, 1 μg/mL pepstin-A and 0.1 μg/mL PMSF) for 30 minutes on ice. In the untreated controls, 100 ng/mL Apo-2L/TRAIL was added after lysis of cells, to immunoprecipitate non-stimulated Apo-2L/TRAIL receptors. One-hundred micrograms (100 μg) of the lysates was incubated at 4° C. for 2 hours with 1 μg each of anti-Apo-2L/TRAIL receptor 1 & 2 (DR4 and DR5) antibodies, provided by Immunex Corp., Seattle Wash. The immune-complexes are incubated overnight at 4° C. with 20 μL of protein A-agarose beads (Roche, Indianapolis, Ind.). The beads are recovered by centrifugation and washed twice with the lysis buffer. The pellet was re-suspended in the sample buffer and analyzed by SDS-PAGE and immunoblot analysis using antibodies against caspase-8, DR5, DR4 and FADD.

Transfection of Dominant-Negative FADD cDNA

Viable Jurkat cells are transfected with the cDNA of dominant negative FADD, which encodes for an 80-208 amino acid death effector domain-containing N-terminus deleted fragment (NFD-4) cloned into pcDNA 3.1 plasmid (Invitrogen Corp., Carlsbad, Calif.) or the control vector (pcDNA 3.1 Zeo), utilizing LipofectAMINE PLUS reagent (Invitrogen Corp.). The transfectants are treated with Apo-2L/TRAIL and/or LAQ824, followed by the evaluation of the percentage of apoptotic cells.

Chromatin Immunoprecipitation (ChIP) Assay

ChIP analysis was performed by a slight modification of a previously described method. Cells are incubated overnight at a density of 0.25×10⁸ cells/ ml at 37° C. with 5% CO₂. Next day, cells are cultured with 0, 50, 100 or 250 nM of LAQ 824 for 24 hours. Formaldehyde was then added to the cells to a final concentration of 1%, and the cells are gently shaken at room temperature for 10 minutes. Following this, the cells are pelleted, suspended in 1 mL of ice-cold PBS containing protease inhibitors (Complete, Boehringer Mannheim). Cells are again pelleted, re-suspended in 0.5 mL of SDS lysis buffer (1% SDS/1.0 mM EDTA/50 mM Tris.HCl, pH 8.1) and incubated on ice for 20 minutes. Lysates are sonicated with 15-second bursts. Debris was removed from samples by centrifugation for 20 minutes at 15,000×g at 4° C. An aliquot of the chromatin preparation (100 μL) was set aside and designated as Input Fraction. The supematants are diluted 3-fold in the immunoprecipitation buffer (0.01% SDS/1.0% Triton X-100/1.2 mM EDTA/16.7 mM Tris.HCl, pH 8.1/150 mM NaCl) and 80 μL of 50% protein A sepharose slurry containing 20 μg sonicated salmon sperm DNA and 1 mg/mL BSA in the TE buffer (10 mM Tris.HCl, pH 8.0/1 mM EDTA) was added and incubated by rocking for 2 hours at 4° C. Beads are pelleted by centrifugation, and supernatants are placed in fresh tubes with 5 μg of the anti-acetylated histone H3 antibody, anti-acetylated histone H4 antibody, or normal rabbit serum and incubated overnight at 4° C. Protein A sepharose slurry (60 μL) was added, and the samples are rocked for 1 hour at 4° C. Protein A complexes are centrifuged and washed 3 times for 5 minutes each with immunoprecipitation buffer and 2 times for 5 minutes each with immunoprecipitation buffer containing 500 mM NaCl. Immune complexes are eluted twice with 250 μL of elution buffer (1% SDS/0.1 M NaHCO3) for 15 minutes at room temperature. Twenty microliters (20 mL) of 5 M NaCl was added to the combined eluates, and the samples are incubated at 65° C. for 24 hours. EDTA, Tris.HCl, pH 6.5 and proteinase K are then added to the samples at a final concentration of 10 mM, 40 mM and 0.04 μg/μL, respectively. The samples are incubated at 37° C. for 30 minutes. Immunoprecipitated DNA (both immunoprecipitation samples and Input) was recovered by phenol/chloroform extraction and ethanol precipitation and analyzed by PCR. DR5 and p21WAF1-specific primers are used to perform PCR on DNA isolated from ChIP experiments and Input samples. The optimal reaction conditions for PCR are determined for each primer pair. For DR5 promoter PCR: forward primer was 5′-GGA GGA MG AGA MG AGA GM AGG MG G-3′ and reverse primer was 5′-TTG GGG GM ATG AGT TGA GGG AGG-3′. The PCR reaction contained 0.2 mM concentration of dATP, dCTP, dGTP and dTTP, 200 nM of each DR5 promoter primer, 1.5 mM of MgCl₂, and 10×PCR buffer containing Tris-HCL (ph 8.0) 500 mM KCL, and 1 U of Tag polymerase (Invitrogen Carlsbad, Calif.). The primer pairs used for p21WAF1 analysis are: 5′-GGT GTC TAG GTG CTC CAG GT-3′ (dp1), 5′-TGTCTAGGTGCTCCAG-3′ (up1). The reactions are performed at 95° C. for 5 minutes, and are followed by 35 cycles of de-naturating at 95° C. for 1 minute, annealing at 56° C. for 1 minute and extension at 72° C. for 1 minute. The PCR products are separated on 2% agarose/eithidium bromide gel. The size of the amplified product was 253 base pairs. The ratio between the immunoprecipitated DNA and Input DNA was calculated for each treatment and primer set. The fold increases after treatment with LAQ824 was calculated from the indicated ratio.

RNase protection assay

A RiboQuant Multi-Probe RNase Protection Assay System was used according to the manufacturer's instructions (BD/PharMingen, San Diego, Calif.). A probe set, hAPO-3d (FLICE, FAS, DR5, DR4 and TRAIL) was used for T7 RNA-polymerase directed synthesis of [α-32P] UTP-labeled antisense RNA probes. The probe set contains the DNA templates, including glyceraldehyde-3-phosphate dehydrogenase (GAPDH) used as internal control. The probes (1×10⁶ cpm/reaction) are hybridized with 20 μg of RNA isolated from the SKW 6.4 and Jurkat leukemia cells, following treatment with 100 nM LAQ824 at different time points using the RNeasy Mini kit (Qiagen, Valencia, Calif.). After overnight hybridization, samples are digested with RNase to remove single-stranded (non-hybridized) RNA. The remaining probes are resolved on 5% de-naturing polyacrylamide gel and analyzed by autoradiography.

RT-PCR Assay for c-FLIP mRNA Levels

Total RNA was isolated from cells utilizing a TRIZOL LS reagent (Invitrogen Carlsbad, Calif.). RT-PCR analysis was performed, as previously described. The RNA (1.0 μg) was reverse-transcribed into cDNA by using SuperScript II RT (Invitrogen Carlsbad, Calif.) according to the manufacturer's protocol. For the c-FLIP PCR, the primer sequences are as follows, forward primer: 5′-GCC CGA GCA CCG AGA CTA CG-3′; and reverse primer: 5′-AGG GAC GGD GAG CTG TGA GAC TG-3′. β-actin forward primer: 5′-CTA CM TGA GCT GCG TGT GG-3′; and reverse primer: MG GM GGC TGG MG AGT GC. The PCR reaction containing 0.2 mM concentration of dATP, dCTP, dGTP, dTTP and 200 nM concentration of each c-FLIP primers and 50 nM of each β-actin primers, 1.5 mM of MgCl₂, and 10×PCR buffer containing Tris-HCL (pH 8.0), 500 mM KCL and 1 U of Tag polymerase (Invitrogen Carlsbad, Calif.). The reaction is performed at 95° C. for 5 minutes, and is followed by 30 cycles of de-naturating at 95° C. for 45 seconds, annealing at 52° C. for 45 seconds and the extension at 72° C. for one minute. The PCR products are separated on a 2% agarose/eithidium bromide gel. The size of the amplified products was 395 bases pairs for the c-FLIP and 527 base pairs for β-actin product, respectively.

Statistical Analyses

Data are expressed as mean ±SEM. Comparisons used student's t-test or ANOVA, as appropriate. P values of <0.05 are assigned significance.

Results

LAQ824 Treatment Induces p21 and p27, as well as Causes Cell Cycle G1 Phase Accumulation and Apoptosis of Jurkat and SKW 6.4 Cells

It has previously been reported that treatment with LAQ824 (5-250 nM) inhibits the in vitro HDAC activity in a dose-dependent manner in the HeLa cell nuclear extracts. Therefore, the effect of LAQ824 on histone acetylation, p21 and p27 levels, as well as on growth arrest and apoptosis of human acute leukemia Jurkat and SKW 6.4 cells is determined. Treatment with 50 nM or 200 nM LAQ824 for 24 hours increased the acetylation of histone H3 and histone H4 in Jurkat and SKW 6.4 cells. LAQ824 mediated histone hyperacetylation is associated with a dose-dependent increase in the levels of p21 in SKW 6.4 but not Jurkat cells. In contrast, although exposure to 50 nM LAQ824 increase the intracellular levels of p27 in both SKW 6.4 and Jurkat cells, treatment with 200 nM of LAQ284 attenuated the p27 levels in both cell-types. These results are consistent with the previous reports that LAQ824 induces the hyperacetylation of nucleosomal histones associated with p21 but not p27 gene promoter, thereby tanscriptionally up-regulating p21 but increasing p27 levels by alternative non-transcriptional mechanism. The effect of LAQ824 on the cell cycle status of SKW 6.4 and Jurkat cells shows that exposure to LAQ824 for 24 hours markedly increases the percentage of cells in the G1 phase and a decline in the S phase of the cell cycle. Importantly, exposure to 10-200 nM of LAQ824 for 24 hours induces apoptosis in a dose-dependent manner, more in Jurkat than in SKW 6.4 cells, as detected by positive staining for annexin V.

LAQ824 Induces DR4, DR5 and Apo-2L/TRAIL but Attenuates the Levels of FLIP, Bcl-2 and IAP Family of Proteins

Based on its ability to induce apoptosis, the effect of LAQ824 on the intracellular levels of the molecular determinants of the extrinsic and intrinsic pathway of apoptosis in SKW 6.4 and Jurkat cells is determined. Exposure to LAQ824 for 24 hours induces Apo-2L/TRAIL, DR4 and DR5 levels. Treatment with LAQ824 attenuates the levels of FLIPL and FLIPS in both SKW 6.4 and Jurkat cells. This is associated with the processing of caspase-9 and -3, suggesting that treatment with LAQ824 not only induces the intrinsic pathway but also primes the cells to the extrinsic pathway induced by Apo-2L/TRAIL. In addition, treatment with LAQ824 also attenuates the levels of Bcl-x_{L}, Bcl-2, XIAP, c-IAP and survivin, which may collectively further lower the threshold to apoptosis due to Apo-2L/TRAIL. In Jurkat cells, these effects of are evident following exposure intervals to LAQ824 of 16 hours or less. Since previous reports have suggested that during apoptosis, several of the determinants of apoptosis belonging to the Bcl-2 and IAP family may be processed by caspases and/or degraded by the proteasome. In Jurkat cells co-treatment with z-VAD-fmk, which inhibits the processing of caspase-3 and PARP, is unable to reverse the attenuating effect of LAQ824 on XIAP, Bcl-2, BCI-X_{L} and c-FLIPL. In addition, co-treatment with the proteasomal inhibitor ALLnL does not restore the levels of XIAP, Bcl-2, c-FLIPL and c-FLIPS attenuated by LAQ824. Whether LAQ824 treatment increases the cell surface expression of DR5, DR4 and Apo-2L/TRAIL is determined. Treatment of Jurkat cells with LAQ824 induces the cell-membrane expression of DR5, as determined by flow cytometry.

LAQ824 Increase the mRNA Levels of DR4 and DR5 but Depletes the mRNA of c-FLIPL

The effect of LAQ824 on the mRNA levels of c-FLIPL, DR5, DR4 and Apo-2L/TRAI, utilizing a multi-probe RNAse protection assay and estimated by densitometry with GAPDH mRNA as the loading control, is determined. Treatment with LAQ824 for 8 hours or 16 hours increased the mRNA expression of DR5 (2.4-fold) and FAS (1.5-fold). DR4 levels increased by 2.2-fold only in SKW 6.4 cells. Exposure to LAQ824 only minimally affected the mRNA levels of Apo-2L/TRAIL and caspase-8 (FLICE). Whether the promoter of DR5 is associated with acetylated histones, which would explain why LAQ824, by causing histone hyperacetylation, would transcriptionally up-regulate DR5 mRNA levels, is determined. The results of the ChIP analyses performed on the lysates of the untreated or LAQ824-treated Jurkat cells shows that treatment with 100 nM and 200 nM LAQ824 for 8 hours increased the level of the DR5 promoter associated with acetylated histones H3 and H4 by 3.3-fold and 5.7-fold, respectively. As has been previously reported, LAQ824 also increased the association of p21WAF1 promoter DNA with acetylated histones in Jurkat and SKW 6.4 cells. In contrast to the increase in the DR5 and DR4 mRNA levels, exposure to LAQ824 for 8 hours inhibits the mRNA level of c-FLIP_{L} by 75%, as determined by an RT-PCR assay. This is reversed by co-treatment with LAQ824 and cyclohiximide (CHX). These results indicate that LAQ824 mediated repression of the c-FLIP_{L} message require new protein synthesis. These results also support the interpretation that LAQ824 augments the levels and activity of a transcriptional repressor for c-FLIP_{L}, an outcome that is neutralized by co-treatment with CHX.

LAQ824 Enhances Apo-2L/TRAIL-Induced DISC Assembly and Activity and Apoptosis

The effects of LAQ824 on Apo-2L/TRAIL-induced DISC and apoptosis is determined, since agents that lower c-FLIP levels and increase DR5 and DR4 levels have been previously shown to enhance Apo-2L/TRAIL-induced DISC activity and apoptosis of leukemia and epithelial cancer cells. Co-treatment with LAQ824 and Apo-2L/TRAIL induces significantly more apoptosis of Jurkat and SKW 6.4 cells, as compared to treatment with either agent alone (p≦0.05). Concomitantly, combined treatment with LAQ824 (20 nM) and Apo-2L/TRAIL (10 ng/mL) for 24 hours, versus treatment with LAQ824 or Apo-2L/TRAIL alone, induces greater processing of caspase-8 and BID, as well as increases processing and the PARP cleavage activity of caspase-3. This involved increased mitochondrial permeability transition, since co-treatment with LAQ824 and Apo-2L/TRAIL, versus LAQ824 or Apo-2L/TRAIL alone, also causes more accumulation of the pro-death molecules cytochrome c, Smac and Omi into the cytosol. To determine the effect of treatment with LAQ824 on Apo-2L/TRAIL-induced DISC, the recruitment of caspase-8, FADD and c-FLIPL into the immunoprecipitates of DR5 and DR4 is compared following treatment with Apo-2L/TRAIL (100 nM for two hours) versus treatment with LAQ824 (100 nM for 24 hours) followed by Apo-2L/TRAIL. Pre-treatment with LAQ824 induces more recruitment of FADD and caspase-8 but not c-FLIPL into the immunoprecipitates of DR4 & DR5, resulting in greater processing of caspase-8 but less of c-FLIPL. To determine whether the increased assembly and activity of DISC due to up-regulation of DR4 and DR5 and down regulation of c-FLIPL and c-FLIPS contributes to enhancement of Apo-2L/TRAIL-induced apoptosis, the effect of the transient transfection of the DED-depleted cDNA of DN-FADD on apoptosis of Jurkat cells induced by Apo-2L/TRAIL or co-treatment with LAQ824 and Apo-2L/TRAIL is determined. As compared to Jurkat cells transfected with the control vector alone (Jurkat-Zeo cells), apoptosis induced by treatment with Apo-2L/TRAIL or by co-treatment with LAQ824 and Apo-2L/TRAIL is inhibited in Jurkat cells transfected with DN-FADD. Importantly, the sensitizing effect of LAQ824 on Apo-2L/TRAIL-induced apoptosis is reduced in Jurkat-DN FADD versus Jurkat-Zeo cells. These findings suggest that LAQ824-induced modulations of the components and activity of Apo-2L/TRAIL-induced DISC contribute toward the overall potentiating effect of LAQ824 on Apo-2L/TRAIL-induced apoptosis.

Combined Treatment with LAQ824 and Apo-2L/TRAIL Overcomes the Inhibition of Apoptosis by Bcl-2 Overexpression

The effects of LAQ824 and/or Apo-2L/TRAIL are compared in HL-60/Bcl-2 cells that possess ectopic over-expression of Bcl-2 (5-fold) versus the control HL-60/Neo cells. LAQ824 mediates increase in p21, p27, DR4 & 5 levels, as well as decline in FLIPL and FLIPS levels are approximately similar, as compared to the untreated in HL-60/Bcl-2 versus HL-60/Neo cells. As has been previously reported, Apo-2L/TRAIL-induced apoptosis was inhibited in HL-60/Bcl-2 versus HL-60/Neo cells. Although following treatment with 50 nM LAQ824 the PARP-cleavage activity of caspase-3 and processing of caspase-8 was also inhibits in HL-60/Bcl-2 cells, exposure to higher level of LAQ824 (100 nM) results in similar processing of PARP and caspase-8 in HL-60/Bcl-2 and HL-60/Neo cells. Additionally, co-treatment with 50 ng/mL of Apo-2L/TRAIL and LAQ824 (50 nM or 100 nM) induces more apoptosis than either agent alone in HL-60/Bcl-2 cells, consistently in over 50% of HL-60/Bcl-2 cells. This is associated with more processing of caspase-8 and BID, with the generation of higher levels of tBID. It is also associated increased PARP cleavage activity of caspase-3 and down regulation of XIAP. These findings suggest that the inhibition of apoptosis due to Apo-2L/TRAIL and lower levels of LAQ824 by Bcl-2 can be overcome by treatment with higher levels of LAQ824 or co-treatment with Apo-2L/TRAIL and LAQ824.

Co-Treatment with LAQ824 Overcomes Resistance to Apo-2L/TRAIL-Induced Apoptosis of Leukemia Blasts from Patients with AML in Relapse

The sensitivity of fresh AML cells procured from patients with relapsed AML to Apo-L/TRAIL and/or LAQ824-induced apoptosis is determined. Table 1 shows that all six samples of AML blasts are resistant to apoptosis induced by Apo-2L/TRAIL (100 ng/mL). In contrast, exposure to LAQ824 (100 nM) induces more apoptosis of the primary AML cells. Co-treatment with LAQ824 and Apo-2L/TRAIL induces more apoptosis than treatment with either agent alone. These data are similar to those derived from HL-60/Bcl-2 cells, in that the resistance of primary AML cells to Apo-2L/TRAIL-induced apoptosis could be overcome by co-treatment with LAQ824 plus Apo-2L/TRAIL. The effect of LAQ824 on the determinants of Apo-2L/TRAIL-induced DISC is determined. In a representative sample of primary AML blasts, and similar to the cultured acute leukemia cells, treatment with 100 nM or 250 nM LAQ824 for 24 hours induces the acetylation of histones H3 and H4. LAQ824 treatment also increases DR4 and DR5 levels, as well as down-regulates the levels of FLIPL and c-FLIPS. Corresponding to the increase in the intracellular levels of DR5 determined by Western analysis, treatment of the primary AML sample with 100 nM and 250 nM LAQ824 also increases the DR5 expression on the cell membrane, as determined by flow cytometry, from a baseline of 17.5% to 33.2 and 62.4% of cells, respectively.

TABLE 1

| | % of Apoptosis | | | |
|---|---|---|---|---|
| | | | | LAQ824 + |
| Patients | Control | LAQ824 | Apo-2L/TRAIL | Apo-2L/TRAIL |
| 1 | 7.0 | 14.5 | 7.6 | 27.2 |
| 2 | 12.0 | 29.5 | 14.0 | 34.5 |
| 3 | 8.0 | 22.9 | 8.6 | 39.4 |
| 4 | 10.0 | 27.9 | 11.0 | 32.3 |
| 5 | 6.0 | 57.8 | 6.8 | 64.9 |
| 6 | 7.0 | 7.9 | 8.1 | 25.4 |

Legend: Co-treatment with LAQ824 enhances Apo-2/L TRAIL-induced apoptosis. Primary AML cells from six patients were treated with LAQ824 (100 nM) and/or Apo-2/L TRAIL (100 ng/mL) for 24 hours. Following this, percentage of apoptotic cells was determined by annexin V staining and flow cytometry. Values represent mean of two experiments performed in duplicate.

What is claimed is:

1. A combination comprising
(a) death receptor ligand, and
(b) a histone deacetylase inhibitor of formula (I) 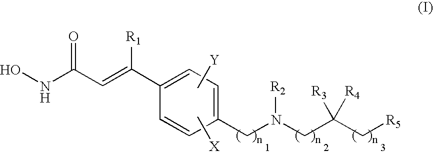
wherein
R₁ is H; halo; or a straight-chain C₁-C₆alkyl, especially methyl, ethyl or n-propyl, which methyl, ethyl and n-propyl substituents are unsubstituted or substituted by one or more substituents described below for alkyl substituents;
R₂ is selected from H; C₁-C₁₀alkyl, preferably C₁-C₆alkyl, e.g., methyl, ethyl or —CH₂CH₂—OH; C₄-C₉cycloalkyl; C₄-C₉heterocycloalkyl; C₄-C₉heterocycloalkylalkyl; cycloalkylalkyl, e.g., cyclopropylmethyl; aryl; heteroaryl; arylalkyl, e.g., benzyl; heteroarylalkyl, e.g., pyridylmethyl; —(CH₂)ₙC(O)R₆; —(CH₂)ₙOC(O)R₆; amino acyl; HON—C(O)—CH═C(R₁)-aryl-alkyl-; and —(CH₂)ₙR₇;
R₃ and R₄ are the same or different and, independently, H; C₁-C₆alkyl; acyl; or acylamino; or
R₃ and R₄, together with the carbon to which they are bound, represent C═O, C═S or C═NR₈; or
R₂, together with the nitrogen to which it is bound, and R₃, together with the carbon to which it is bound, can form a C₄-C₉heterocycloalkyl; a heteroaryl; a polyheteroaryl; a non-aromatic polyheterocycle; or a mixed aryl and non-aryl polyheterocycle ring;
R₅ is selected from H; C₁-C₆alkyl; C₄-C₉cycloalkyl; C₄-C₉heterocycloalkyl; acyl; aryl; heteroaryl; arylalkyl, e.g., benzyl; heteroarylalkyl, e.g., pyridylmethyl; aromatic polycycles; non-aromatic polycycles; mixed aryl and non-aryl polycycles; polyheteroaryl; non-aromatic polyheterocycles; and mixed aryl and non-aryl polyheterocycles;
n, n₁, n₂ and n₃ are the same or different and independently selected from 0-6, when n₁ is 1-6, each carbon atom can be optionally and independently substituted with R₃ and/or R₄;
X and Y are the same or different and independently selected from H; halo; C₁-C₄alkyl, such as CH₃ and CF₃; NO₂; C(O)R₁; OR₉; SR₉; CN; and NR₁₀R₁₁;
R₆ is selected from H; C₁-C₆alkyl; C₄-C₉cycloalkyl; C₄-C₉heterocycloalkyl; cycloalkylalkyl, e.g., cyclopropylmethyl; aryl; heteroaryl; arylalkyl, e.g., benzyl and 2-phenylethenyl; heteroarylalkyl, e.g., pyridylmethyl; OR₁₂; and NR₁₃R₁₄;
R₇ is selected from OR₁₅; SR₁₅; S(O)R₁₆; SO₂R₁₇; NR₁₃R₁₄; and NR₁₂SO₂R₆;
R₈ is selected from H; OR₁₅; NR₁₃R₁₄; C₁-C₆alkyl; C₄-C₉cycloalkyl; C₄-C₉heterocycloalkyl; aryl; heteroaryl; arylalkyl, e.g., benzyl; and heteroarylalkyl, e.g., pyridylmethyl;
R₉ is selected from C₁-C₄alkyl, e.g., CH₃ and CF₃; C(O)-alkyl, e.g., C(O)CH₃; and C(O)CF₃;
R₁₀ and R₁₁ are the same or different and independently selected from H; C₁-C₄alkyl; and —C(O)-alkyl;
R₁₂ is selected from H; C₁-C₆alkyl; C₄-C₉cycloalkyl; C₄-C₉heterocycloalkyl; C₄-C₉heterocycloalkylalkyl; aryl; mixed aryl and non-aryl polycycle; heteroaryl; arylalkyl, e.g., benzyl; and heteroarylalkyl, e.g., pyridylmethyl;
R₁₃ and R₁₄ are the same or different and independently selected from H; C₁-C₆alkyl; C₄-C₉cycloalkyl; C₄-C₉heterocycloalkyl; aryl; heteroaryl; arylalkyl, e.g., benzyl; heteroarylalkyl, e.g., pyridylmethyl; amino acyl; or
R₁₃ and R₁₄, together with the nitrogen to which they are bound, are C₄-C₉heterocycloalkyl; heteroaryl; polyheteroaryl; non-aromatic polyheterocycle; or mixed aryl and non-aryl polyheterocycle;
R₁₅ is selected from H; C₁-C₆alkyl; C₄-C₉cycloalkyl; C₄-C₉heterocycloalkyl; aryl; heteroaryl; arylalkyl; heteroarylalkyl; and (CH₂)ₘZR₁₂;
R₁₆ is selected from C₁-C₆alkyl; C₄-C₉cycloalkyl; C₄-C₉heterocycloalkyl; aryl; heteroaryl; polyheteroaryl; arylalkyl; heteroarylalkyl; and (CH₂)ₘZR₁₂;
R₁₇ is selected from C₁-C₆alkyl; C₄-C₉cycloalkyl; C₄-C₉heterocycloalkyl; aryl; aromatic polycycles; heteroaryl; arylalkyl; heteroarylalkyl; polyheteroaryl and NR₁₃R₁₄;
m is an integer selected from 0-6; and
Z is selected from O; NR₁₃; S; and S(O),
or a pharmaceutically acceptable salt thereof.

2. A method for the prevention or treatment of proliferative diseases, in a mammal, which comprises treating the mammal with pharmaceutically effective amounts of a combination of:
(a) death receptor ligand, and
(b) a histone deacetylase inhibitor of formula (I) according to claim 1.

3. The combination according to claim 1, wherein the death receptor ligand is TRAIL, TRAIL/Apo-2L, TRAIL mimetics, agonistic antibodies, or other agents that can bind to DR4 and DR5 triggering the activity of caspase-8 and apoptosis through the assembly of a cell-membrane associated multi-protein death inducing signaling complex (DISC).

4. The combination of claim 1, wherein the HDAI is selected from the group consisting of N-hydroxy-3-[4-[[(2-hydroxyethyl)[2-(1H-indol-3-yl)ethyl]-amino]methyl]phenyl]-2E-2-propenamide, N-hydroxy-3-[4-[[[2-(1H-indol-3-yl)ethyl]-amino]methyl]phenyl]-2E-2-propenamide and N-hydroxy-3-[4-[[[2-(2-methyl-1H-indol-3-yl)-ethyl]-amino]methyl]phenyl]-2E-2-propenamide, or a pharmaceutically acceptable salt thereof.

5. The combination of claim 1 for the prevention or treatment of leukemia.

6. The method of claim 2, wherein the mammal is a human.

7. The combination of claim 1 for the prevention or treatment of acute myeloid leukemia (AML).

8. A combined preparation which comprises:
(a) one or more unit dosage forms of a death receptor ligand; and
(b) one or more unit dosage forms of a HDAI of formula (I) of claim 1.

9. The combined preparation according to claim 8, wherein the death receptor ligand is TRAIL, TRAIL/Apo-2L, TRAIL mimetics, agonistic antibodies, or other agents that can bind to DR4 and DR5 triggering the activity of caspase-8 and apoptosis through the assembly of a cell-membrane associated multi-protein DISC.

10. The combined preparation of claim 9, wherein the histone deacetylase inhibitor is selected from the group consisting of N-hydroxy-3-[4-[[(2-hydroxyethyl)[2-(1H-indol-3-yl)ethyl]-amino]methyl]phenyl]-2E-2-propenamide, N-hydroxy-3-[4-[[[2-(1H-indol-3-yl)-amino]methyl]phenyl]-2E-2-propenamide and N-hydroxy-3-[4-[[[2-(2-methyl-1H-indol-3-yl)-ethyl]-amino]methyl]phenyl]-2E-2-propenamide, or a pharmaceutically acceptable salt thereof.

11. A method of treating or preventing premalignant proliferative diseases in a mammal which comprises treating the mammal with a combination of:
(a) a pharmaceutically effective amount of a death receptor ligand; and
(b) a pharmaceutically effective amount of N-hydroxy-3-[4-[[(2-hydroxyethyl)[2-(1H-indol-3-yl)ethyl]-amino]methyl]phenyl]-2E-2-propenamide, N-hydroxy-3-[4-[[[2-(1H-indol-3-yl)ethyl]-amino]methyl]phenyl]-2E-2-propenamide or N-hydroxy-3-[4-[[[2-(2-methyl-1H-indol-3-yl)-ethyl]-amino]methyl]phenyl]-2E-2-propenamide; or a pharmaceutically effective salt thereof.

12. The method according to claim 11, wherein the death receptor ligand is TRAIL, TRAIL/Apo-2L, TRAIL mimetics, agonistic antibodies, or other agents that can bind to DR4 and DR5 triggering the activity of caspase-8 and apoptosis through the assembly of a cell-membrane associated multi-protein DISC.

13. A method of treating or preventing proliferative diseases in a mammal which comprises treating the mammal with a combination of:
(a) a pharmaceutically effective amount of a death receptor ligand; and
(b) a pharmaceutically effective amount of an HDAI.

14. A combined preparation which comprises:
(a) one or more unit dosage forms of a death receptor ligand; and
(b) one or more unit dosage forms of a HDAI.
